(19) United States
(12) Patent Application Publication
(10) Pub. No.: US 2006/0222694 A1
(43) Pub. Date: Oct. 5, 2006

(54) Stabilized topotecan liposomal composition and methods

(76) Inventors: Choon K. Oh, Collegeville, PA (US); Francis Ignatious, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/560,693

(22) PCT Filed: Jun. 25, 2004
(86) PCT No.: PCT/US04/20592

§ 371 Date: Mar. 30, 2006

Related U.S. Application Data
(60) Provisional application No. 60/483,410, filed on Jun. 27, 2003.
(60) Provisional application No. 60/530,163, filed on Dec. 17, 2003.

(51) Int. Cl. A61K 9/127

(52) U.S. Cl. 424/450; 977/907

(57) ABSTRACT

A topotecan liposomal composition which can be reconstituted from a lyophilized form to an injectable liposome suspension having selected liposome sizes in the size range between 0.05 and 0.25 microns, and between about 85-100% liposome-entrapped topotecan is disclosed.

### FIELD OF THE INVENTION

The present invention relates to a novel stable topotecan liposomal composition, and in particular, to a composition which can be stored for extended periods in lyophilized form without significant topotecan degradation. This invention also relates to a reconstituted topotecan liposomal composition having selected liposome sizes and between 85-100 percent liposome-entrapped topotecan. This invention also relates to processes for preparing the above-described compositions.

### BACKGROUND OF THE INVENTION

The present invention provides a stable lyophilized liposomal composition for camptothecin analogues, such as topotecan, which are prone to chemical degradation during storage in aqueous media present in conventional liposome suspensions. In particular, the invention provides a lyophilized liposome solid composition in which a camptothecin analogue, such as topotecan, can be entrapped within the liposome interior, and which upon reconstitution with aqueous media, and optionally with further dilution, provides a liposome suspension composition in a form that is ready to be administered by intravenous (parenteral) administration.

Camptothecin analogues, such as (S)-10-[(dimethylamino)methyl-4-ethyl-4,9-dihydroxy-1H-pyrano [3′,4′: 6,7]indolizino[1,2-b]quinolone-3,14(4H, 12H)dione monohydrochloride, commonly known as topotecan hydrochloride, have demonstrated usefulness as both antineoplastic and antiviral therapeutic agents.

One problem with camptothecin is its water insolubility, which hinders the delivery of the drug. Numerous analogues of camptothecin have been prepared to improve the compound's water solubility. Topotecan is a semi-synthetic water-soluble analog of camptothecin which is an inhibitor of topoisomerase 1. Topotecan, like other camptothecin analogs, stabilizes the covalent complex between topoisomerase I and DNA, resulting in enzyme-linked DNA cleavage and single-strand breaks.

Topotecan HCl for Injection (Hycamtin®, GlaxoSmithKline) has been approved as safe and effective by the United States Food and Drug Administration for second line therapy for refractory ovarian cancer. One current therapy for Hycamtin (topotecan hydrochloride) comprises administration of a dose of 1.5 mg/m² of topotecan by intravenous infusion over 30 minutes daily for 5 consecutive days, starting on day 1 of a 21-day course. One drawback of this frequent parenteral administration is patient discomfort. Another drawback is that parenteral administration requires the patient to travel to the physician's office resulting in patient inconvenience. Thus, there is a need to develop a sustained release formulation of topotecan that would allow dosing regimens which would minimize inconvenience and discomfort to the patient.

Another problem with camptothecin and its analogues, including topotecan, is that the compounds are susceptible, in aqueous environments, to hydrolysis at the α-hydroxy lactone ring. The lactone ring opens to the carboxylate form of the drug, a form that exhibits little activity against topoisomerase 1. Various approaches to improving the lactone ring stability of camptothecin and its analogues have been described. For example, it is now known that the hydrolysis of the lactone moiety can be reduced if the compound is maintained in a low pH environment. Another approach has been to entrap the compounds in liposomes. Several camptothecin/liposome compositions have been described (Constantinides et al. (WO 95/08986); Burke (U.S. Pat. No. 5,552,156); Moynihan (WO9913816); Slater (U.S. Pat. No. 6,355,268), and Madden (US20020119990), each of which are incorporated herein by reference in their entirety).

Although the encapsulation of camptothecin analogues, particularly topotecan, within liposomes having an acidic interior, may improve the stability of the lactone ring, such encapsulation does not prevent other undesirable chemical degradation of the topotecan in aqueous media. Kearney et al. (International Journal of Pharmaceutics, 127, (1996), 229) report that topotecan in aqueous media undergoes a pH and temperature dependent degradation, proceeding through the deamination of the side chain, resulting in the formation of 10-hydroxy camptothecin and a camptothecin dimer. These degradation products, especially the capmptothecin dimer, are practically insoluble in water, and precipitate as needle shaped crystals. When a liposomal topotecan suspension formulation was stored for a few months, the number and size of the crystals surpassed the foreign particulate matter permitted by the USP <788> testing. One of the ways of overcoming the degradation of the topotecan is to lower the pH of the storage medium to less than 3. In conventional liposomal formulations, it may not be possible to provide such a low pH environment inside or outside the liposomes. Problematically, the lipid components of the liposomes have questionable storage stability in such low pH environments.

Another possible approach is to dehydrate the liposomes by lyophilization. Lyophilization of such drug-encapsulated liposomal compositions, followed by reconstitution to form an administerable dosage form has been attempted. However, conventional lyophilization and reconstitution processes have resulted in substantial release of free (unencapsulated) drug on reconstitution. For example, conventional lyophilization and reconstitution procedures result in a substantial release of liposome entrapped drug, typically 20-30% of free (unencapsulated) drug is detected in the reconstituted liposome suspension. (T. Madden, Chapter 11, “Liposomes Rational Design” Ed, A. S. Janoff, Marcel Dekker, New York 1999). Accordingly, there remians a need to develop a camptothecin composition that provides the benefits of liposomal formulations that can maintain stability upon storage.

### SUMMARY OF THE INVENTION

This invention is directed to a stable, lyophilized liposomal topotecan composition comprising topotecan, liposomes and cryprotectant, wherein the topotecan is substantially entrapped within the liposome interior. Specifically, at least about 85% of the topotecan in the composition is liposome-entrapped topotecan, and wherein about 85% of the liposomes in said composition have sizes of about 0.05 to about 0.25 microns. This invention is also directed to a reconstituted liposomal topotecan composition comprising topotecan, liposomes and cryprotectant, wherein about 85% of the liposomes in said composition have sizes of about 0.05 to about 0.25 microns, at least about 85% of the topotecan in the composition is liposome-entrapped topotecan, and the topotecan/lipid ratio is about 0.02 to about 0.4 or about 2 to 40 percent by weight, wherein the lipid is a mixture of cholesterol and a vesicle-forming lipid (e.g., a phospholipid or mixture of phospholipids), which forms the liposome. In another embodiment, this invention is directed to a process for preparing an aqueous liposomal topotecan composition suitable for injection, wherein the injectable composition is comprised of at least about 85% liposome-entrapped topotecan. This process comprises reconstituting the above-described lyophilized composition in an aqueous medium. In another embodiment, this invention is directed to a lyophilized liposomal topotecan which can be stored for extended periods of time, without causing significant topotecan degradation, as attested by the USP <788> particulate matter testing performed on the reconstituted lyophile. In yet another embodiment, this invention is directed to a two-step reconstitution process, wherein the above-described lyophilized composition is first reconstituted to a form a concentrate composition which is near physiological osmolarity, and diluting the concentrate composition with an aqueous medium to provide an aqueous composition suitable for intravenous injection.

### BRIEF DESCRIPTION OF THE DRAWINGS

FIG. 1 is a micrograph image of Cryo-TEM liposomal topotecan suspension formulation before lyophilization.

FIG. 2 is a micrograph image of Cryo-TEM liposomal topotecan (reconstituted suspension) after lyophilization.

### DETAILED DESCRIPTION OF THE INVENTION

In one embodiment, this invention provides a liposomal topotecan formulation which has selected liposome size characteristics, is stable on long-term storage, and contains at least about 85% topotecan in liposome-encapsulated form. In another embodiment, this invention provides a lyophilized liposomal topotecan composition, in which the majority of topotecan inside the liposomes is in the precipitated state. In yet another embodiment, this invention provides a process for preparing a lyophilized liposomal topotecan which, after long-term storage, and subsequent reconstitution provides a ready to use liposome composition which has preselected liposome sizes, relatively little unencapsulated topotecan, and which passes USP <788> particulate matter testing. In another embodiment, this invention provides a lyophilized liposomal topotecan composition which, upon reconstitution with a predetermined volume of aqueous medium, provides a liposome concentrate characterised by liposome sizes predominantly in a selected size range between about 0.05 to 0.25 microns and liposome-encapsulated topotecan, at a topotecan/lipid ratio between 0.02 and 0.4 or about 2-40 percent by weight, and containing about 85%-100% of the total topotecan. In yet another embodiment, this invention provides a reconstituted topotecan liposomal composition, reconstituted with a low-osmolarity medium or distilled water that provides a concentrate which is near physiological osmolarity

I. Liposomal Topotecan Preparation

The process for the preparation of the liposomal topotecan suspension used for the preparation of the lyophile consists of two steps: the first involves the preparation of empty liposomes, and the second consists of loading topotecan into the empty liposomes using a remote loading technique.

A. Empty Liposome Preparation

The empty liposome suspension can be prepared using a variety of processes, as described in, e.g., Szoka, et al., Ann. Rev. Biophys. Bioeng. 9:467 (1980); U.S. Pat. Nos. 4,235,871; 4,501,728; 4,837,028, “Liposomes,” Marc J. Ostro, ed., Marcel Dekker, Inc., New York, 1983, Chapter 1; and Hope, et al., Chem. Phys. Lip. 40:89 (1986), all of which are incorporated herein by reference. The protocol for generating liposomes generally includes: mixing of lipid components in an organic solvent; drying and resuspending the lipids in an aqueous buffer solution; and sizing of liposomes (such as by extrusion), all of which are well known in the art. Such buffer solutions may be selected to have a predetermined pH and contain predetermined salts and/or other constituents such as sucrose, as is well known in the art.

Alternative processes of preparing liposomes are also available. For instance, a process involving detergent dialysis based self-assembly of lipid particles is described in Wheeler, et al., U.S. Pat. No. 5,976,567. Another process of producing empty liposomes is by solvent injection in which a solution of liposome lipids in a selected solvent system is injected, at a selected rate, into a mixing chamber containing an aqueous medium containing cryoprotectant(s), salt(s), and buffer constituents at a predetermined pH. The lipid solution as well as the aqueous medium is typically maintained at a temperature higher than the main chain gel-to-liquid crystallisation transition temperature, Tm, of the lipids. The liposome dispersion being formed in the mixing chamber may then be sized by several passages through a sizing apparatus, e.g. a Lipex Biomembranes extruder (Northern Lipids, Vancouver, Canada), designed to size the liposomes to a particular diameter range. The apparatus may contain a polycarbonate membrane or ceramic filter for sizing purposes. The liposomes produced may then be subjected to a diafiltration process to exchange the extra liposome medium with a medium suitable for the drug loading process. A preferred cryoprotectant of this invention is sucrose, trehalose or lactose, at a concentration of from about 5% to about 20%, specifically about 5% to about 15% of the liposome suspension, prior to lyophilization.

B. Lipid Components

The empty liposomes can be formed from cholesterol and/or standard vesicle-forming lipids, which generally include neutral phospholipids, such as phosphatidyl cholines (PC), including those obtained from egg, soy beans or other plant sources or those that are partially or wholly synthetic, of variable lipid chain length and unsaturation and which are suitable for use in the present invention.

Synthetic, semisynthetic and natural product sphingomyelins, phosphatidyl cholines including, but not limited to, distearoylphosphatidyl choline (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. In one embodiment of this invention, hydrogenated soy phosphatidylcholine and/or distearoylphosphatidylcholine are used as the phosphatidylcholine. Preferably, hydrogenated soy phosphatidylcholine is the phosphatidylcholine.

Phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, and phosphatidic acids containing lauric, myristic, stearic, and palmitic acid chains; negatively charged phospholipids, such as phosphatidylglycerol (PG), phosphatidylserine (PS), phosphatidylinositol (PI) and phosphatidic acid (PA); negatively charged sterols, such as cholesterol sulfate and cholesterol hemisuccinate; and sterols such as cholesterol. The lipid used may be conjugated to a hydrophilic polymer such as polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and polyglycerol. The selection of lipids is generally guided by consideration of, e.g., liposome size and stability of the liposomes in the bloodstream and shelf-life stability.

Preferred liposome compositions for use in the present invention include those comprising sphingomyelin and cholesterol. The ratio of sphingomyelin to cholesterol in the liposome composition can vary, but generally is in the range of from about 75/25 mol %/mol % sphingomyelin/cholesterol to about 30/70 mol %/mol % sphingomyelin/cholesterol. In one embodiment, liposome compositions containing about 70/30 mol %/mol % sphingomyelin/cholesterol to about 55/45 mol %/mol % sphingomyelin/cholesterol can be used. In yet another embodiment, liposome compositions containing about 55/45 mol %/mol % sphingomyelin/cholesterol can be used. Other lipids can be included in the liposome compositions of the present invention as may be necessary, such as to prevent lipid oxidation or to attach ligands onto the liposome surface. Generally, if other lipids are included, the inclusion of such lipids will result in a decrease in the sphingomyelin/cholesterol ratio. Liposomes of this type are known as sphingosomes and are more fully described in U.S. Pat. No. 5,814,335, the disclosure of which is incorporated herein by reference.

C. Loading of Empty Liposomes with Camptothecin Analogues

Preparation of liposomal entrapped camptothecins requires loading of the camptothecin drug into the liposomes. Loading can be either passive or active. Passive loading generally requires addition of the drug to the above-described buffer solution at the time of reconstitution/hydration. During the loading process, the drug becomes trapped within the aqueous interior of the liposome, where it will predominantly remain if it is not lipid soluble, and if the vesicle remains intact (such processes are employed, for example, in PCT Publication No. WO 95/08986, the disclosure of which is incorporated herein by reference).

Active loading is in many ways preferable, and a wide variety of therapeutic agents can be loaded into liposomes with encapsulation efficiencies approaching 100% by using a transmembrane pH or ion gradient (see, Mayer, et al., Biochim. Biophys. Acta 1025:143-151 (1990) and Madden, et al., Chem. Phys. Lipids 53:37-46 (1990)). Numerous ways of active loading are known to those of skill in the art. All such methods involve the establishment of some form of transmembrane gradient (for example, a concentration gradient or a pH gradient) that draws therapeutic compounds into the interior of liposomes where they can reside for as long as the transmembrane gradient is maintained. Very high quantities of the desired drug can be loaded into the liposome interior, to the extent that the drug may exceed its aqueous solubility limit and precipitate out of solution in the liposome interior allowing continuous drug uptake down its concentration gradient.

Particularly preferred for use with the instant invention is an ionophore mediated drug loading process as described in U.S. Pat. No. 5,837,282, the disclosure of which is incorporated by reference herein. This method employs an ionophore in the liposome membrane to drive the generation of a transmembrane pH gradient from a previously existing transmembrane monovalent or divalent ion gradient.

Another preferred method of active loading uses a transmembrane ammonium ion gradient as described in U. S. Pat. Nos. 5,316,771 and 5,192,549. Still another preferred method of loading amphipathic drug substances is described in U.S. Pat. No. 5,785,987, wherein an alkylammonium gradient is used for loading amphiphilic drug substances.

Another preferred method of active loading uses a monomeric or polymeric multivalent acid inside the liposomes, such that a co-precipitate is formed with topotecan, that has been loaded via a transmembrane gradient method, and the precipitation process acts as the driving force for further loading of topotecan into the liposomes. The multivalent acid may be polymeric or non-polymeric, organic or inorganic. For example, the multivalent acid may be a polysulfate, polysulfonate, polyphosphate or polycarboxylate such as tartrate, citrate, sulfate, phosphate, diethylene thiamine pentacetate, or polyacrylate. In this embodiment, the precipitating agent may be polymeric or non-polymeric. The polymeric compounds may be, for example, polyacrylate, chondroitin sulfate A, dextran sulfate, polyvinyl sulfuric acid, or polyphosphoric acid.

It is recognized that in all the methods of active loading of topotecan within the liposomes, the counter ion present inside the liposomes is selected in such way as to precipitate the topotecan as it migrates to the interior of the liposomes. For example, in the ionophore mediated loading process, a MgSO₄ solution is present inside the liposomes, such that as the Mg ions are transported by ionophore, S04 (sulfate) anions are left within the liposomes. Topotecan, while it is being loaded into the liposomes, combines with the free sulfate anions and forms a precipitate of topotecan sulfate. Similarly, in the case of liposomal loading with either a transmembrane ammonium or alkyl ammonium gradient, ammonium sulfate or alkyl ammonium sulfate, are selected such that topotecan can form precipitates of topotecan sulfate inside the liposomes.

II. Lyophilization of the Liposome Suspension.

Once the liposomal topotecan suspension is prepared, it is lyophilized. During the lyophilization process, the liposome structure could become damaged leading to leakge of the encapsulated topotecan. Such damage can be prevented by the use of cryoprotectants, which may be present in certain predetermined ratios with respect to the lipid concentration. These cryoprotectants are present both in the internal as well as external medium of the liposomes.

These cryoprotectants may be selected from sugars such as sucrose, trehalose, lactose, maltose, cyclodextrin and its derivatives.

These cryoprotectants may also be polymeric such as polyethylene glycol, dextran, polyvinylpyrrolidone, or hydroxyethyl starch.

These cryoprotectants may be used alone or as a combination.

The cryoprotectants are introduced into the intraliposomal aqueous layer during the preparation of empty liposomes by using these cryoprotectants dissolved in the hydration media. Externally, the cryoprotectants are introduced during the diafiltration performed after the completion of the drug loading process. The desired cryoproectant may also be introduced by the exchange of the external buffer of any liposomal suspenion formulation by diafiltration.

The liposomal suspension is filled into vials and lyophilized.

During the lyophilization process the liposomal suspension is first frozen and the frozen mass is subjected to a primary drying step at a suitable temperature and vacuum, such that ice is sublimed under vacuum without collapsing the frozen mass. Typically, the shelf temperature for primary drying may be varied within a range of −18 to −36 ° C., at suitable pressure ranging from 0 to 250 mTorr. The formulation, size, shape of the vial, number of vials and type of lyophilizer will control the time required to complete primary drying, which may vary from few hours up to several days. Upon completion of primary drying the shelf temperature is raised to the desired setting to perform secondary drying. Typically, the shelf temperature may be varied within a range of 0 to 30° C., at suitable pressure ranging from 0 to 250 mTorr. The duration of secondary drying is dictated by the level of residual moisture acceptable for the final product. Typically, the secondary drying may last for a few hours to several hours.

The residual moisture level in the lyophilized product impacts the storage stability of the lyophile at a desired temperature and duration. Desirably, the amount of residual moisture in the lyophile should be less than 6%, more preferably less than 4%.

III. Reconstitution of the Lyophile

At a desired stage, typically before administration to the patient, the lyophile which has can stored for an extended period of time at a predetermined temperature, needs to be reconstituted with an appropriate medium to produce a liposome suspension. The reconstitution medium may include sterile water, water for injection, a pH buffered solution, or 5% dextrose solution (D5W). The reconstitution is usually performed at room temperature, however other temperatures may also be considered.

The reconstituted lyophiles of this invention are comprised of:

a. liposomes having liposome sizes predominantly (about 85%) in a selected size range between about 0.05 to 0.25 microns,

b. liposome-entrapped topotecan, at a topotecan/lipid weight ratio between about 0.02 and 0.4, and between about 85-100% liposome-entrapped topotecan; and

c. liposome suspension which passes the USP <788> particulate matter test.

The USP particulate matter test defines the number of foreign particulate matter as observed by optical microscopy. As per USP <788>, the limit for foreign particulate matter having size greater than or equal to 10 microns is 3000, and for particles having size greater than or equal to 25 microns is 300.

The reconstituted suspension of this invention demonstrates minimal chemical degradation, both in terms of lipids and topotecan.

An advantage of the lyophilized compositions of this invention is that they can be stored long term at refrigerator or room temperature without appreciable lipid degredation (oxidative or hydrolytic), and with minimal chemical degradation of topotecan.

The topotecan liposome lyophilized composition can also be readily reconstituted to a concentrate having a desired liposome size distribution and at least about 85%, and more typically, 90-100 liposome-entrapped topotecan. The topotecan liposome concentrate may be diluted for parenteral administration without significantly changing liposome size or percentage of liposome-bound drug. The reconstituted topotecan liposome composition of this invention, prepared using either a one step or two step (by diluting a concentrate) process has advantageous biodistribution properties, due to the size distribution of liposomes, to the integrity of both the liposome and topotecan components, and to the low percentage of unencapsulated topotecan.

### EXAMPLES

The following examples illustrate methods of preparation and properties of the liposomal topotecan suspensions, lyophilized powder and reconstituted suspensions formed according to this invention. The examples are in no way intended to limit the scope of the invention.

Materials

Topotecan HCl was obtained from GlaxoSmithKline; Egg Sphingomyelin and Cholesterol, from Avanti Polar Lipids, Inc., (Birmingham, Ala.); sucrose, trehalose dihydrate, lactose, magnesium sulfate, sodium phosphate monobasic, sodium phosphate dibasic, ethylenediamine tetraacetic acid sodium salt, and calcimycin from Sigma Chemical (St. Louis, Mo.); 200 proof ethanol from AAPER Alcohol and Chemical Co. (Shelbyville, Ky.).

### Example 1

Preparation of liposomal entrapped topotecan suspensions can be conducted on large scale (e.g., >100 g) or small scale (e.g., <100 g). Methods of active loading useful for the preparation of liposomal entrapped topotecan suspensions are described in U.S. Pat. Nos. 5,837,282, 5,316,771, 5,192,549, 5,785,987, 6,355,268, 6,465,008, and U.S. Pat. Appln. Pub. Nos. 2002011990 and 20020110586. Exemplified below is a process for a lab-bench scale preparation of a liposomal entrapped topotecan suspension.

Preparation of Empty Liposomes

An ethanolic (7.5 mL) solution containing 0.6 g cholesterol and 1.3 g sphingomyelin is prepared at 60° C. This warm ethanolic solution is injected into 50 mL of hydration buffer thermostatted at 60° C. with continuous stirring. The hydration buffer is comprised of 353 mM MgSO₄ and 235 mM sucrose. The multilamellar liposome suspension is sized by extrusion under nitrogen pressure using a 100 mL thermobarrel extruder thermostatted at 65° C. The extruder is stacked with two (nucleopore) polycarbonate membranes having equal pore diameters of 80 nm. The extrusion process is repeated until a desired mean particle size of 110 nm (approximately) is obtained. The resulting liposome suspension is cooled to 35° C., and subjected to diafiltration using a buffer consisting of 300 mM sucrose and 10 mM sodium phosphate at pH 6, in order to remove residual ethanol and MgSO₄ present on the outside of the liposomes. The concentration of the empty liposome suspension is adjusted to 40 mg/mL of lipids.

Loading of Topotecan Into Empty Liposomes

To an incubated (60° C.) mixture of 10 mL of empty liposomes (conc.: 40 mg/mL of lipids), 2.6 mL of 250 mM EDTA sodium pH adjusted to 6, 1.3 mL 1M sodium phosphate buffer at pH 6, and 0.1 mL calcimycin at a concentration of 2mg/mL, is added a solution containing 4 mL of a topotecan solution (10 mg/mL) prepared in 300 mM sucrose/tartaric acid buffer (1 mg/mL). The resulting mixture is maintained at the incubation temperature for 30 min (the loading process), then is cooled to 15° C. The resulting loaded liposomes are subjected to diafiltration using 300 mM sucrose/10 mM sodium phosphate (pH 6) to remove the unencapsulated topotecan as well as other processing aids used for loading. Once the washings are completed, the concentration of the liposomal topotecan suspension is adjusted to 40 mg /mL lipid (approximately 4 mg/mL topotecan).

Alternatively, the 300 mM sucrose/10 mM sodium phosphate (pH 6) buffer may be replaced with a 300 mM trehalose/10 mM sodium tartrate buffer (pH 4) during the diafiltration process.

### Example 2

Lyophilization of Liposomal Topotecan

One mL samples of a liposomal topotecan suspension composition (containing 4 mg/mL topotecan) were filled into 3 mL vials for lyophilization. Lyophilization was conducted using a Lyostar II freeze dryer (sold by FTS Kinetics, New York).

Lyophilization Cycle Used

Freezing conditions

| Shelf Temperature | −40° C. |
|---|---|
| Ramp rate | 2.5° C./min |
| Hold time | 15 min |
| Final Freeze set point | −40° C. |
| Extra freeze time | 60 min |
| Vacuum start set point | 20 |

Primary Freeze Drying

| | |
|---|---|
| Shelf set point | −10° C. |
| Ramp Rate | 2.5 |
| Time | 900 min |
| Vacuum set point | 100 milliTorr |

Secondary Drying

| | |
|---|---|
| Shelf set point | 0° C. |
| Ramp rate | 0.5 |
| Time | 540 min |
| Vacuum set point | 100 milli Torr |

Final Hold

| | |
|---|---|
| Shelf set point | 5° C. |
| Time | 15 min |
| Vacuum set point | 100 milli Torr |

Another set of liposomal topotecan samples containing sucrose in the external medium in one case and trehalose in another were lyophilized using the same lyophilization cycle as above, except for the primary drying temperature, which was set at −30° C. Table 1 summarises the experiments.

TABLE 1

| | | | Reconstitution with |
|---|---|---|---|
| | Primary drying | Moisture | 1 mL milliQ |
| Expt. No. | Temperature | Content | water |
| in sucrose buffer | −10° C. | 3.8% | Readily forms liposomes |
| | | | without shaking |
| in sucrose buffer | −10° C. | 6.1% | Readily forms liposomes |
| | | | without shaking |
| in trehalose buffer | −30° C. | NA | Readily forms liposomes |
| | | | without shaking |
| in trehalose buffer | −30° C. | NA | Readily forms liposomes |
| | | | without shaking |

### Example 3

Reconstitution and Analysis of the Reconstituted Products.

The lyophiles from Example 2 were obtained as a yellowish cake, which reconstituted easily without shaking to form liposome suspensions. The samples were reconstituted using 1 mL purified (milliQ) water.

The particle size of the original liposome suspensions before lyophilization, and the corresponding product after reconstitution of the lyophile were measured by dynamic light scattering methods using a Nicomp 380 particle size analyzer. The total topotecan present in the samples were determined using an HPLC method, after dissolving the liposomal topotecan samples in methanol. The unencapsulated topotecan in the liposomal topotecan samples was isolated using a 30 KD Micron filter, and the quantified using the sample HPLC assay. The data obtained are shown in Table 2. According to the data presented in the Table, lyophilization and reconstitution retains the particle size of the original liposome sample. It was observed that more than 90% of the topotecan was retained in the liposomes during lyophilization.

TABLE 2

| | Unencapsulated | | | Mean |
|---|---|---|---|---|
| | Topotecan (% | Osmolality | | Particle Size |
| Expt. No. | total) | (mmol/Kg) | pH | (nm) |
| in sucrose | empty liposome | 298 | 5.7 | 107 (std dev. 31)/ |
| Buffer | | | | |
| in sucrose | 6.2/0.2* | 320 | 5.7 | 121 (std dev 37)/ |
| buffer | | | | 115 (29)* |
| in trehalose | 4.7/0.3* | NA | NA | 112 (std dev. 34)/ |
| buffer | | | | 110 (21)* |
| in trehalose | 7.0/0.6* | NA | NA | 118(std.dev.30)/ |
| buffer | | | | 109 (9)* |

*Data for the samples before lyophilization

### Example 4

Lyophilization of Liposomal Topotecan

One mL samples of a liposomal topotecan suspension formulation (containing 4 mg/mL topotecan) were filled into 3 mL vials for lyophilization. Lyophilization was conducted using a Lyostar II freeze dryer (sold by FTS Kinetics, New York)

Lyophilization Cycle Used

Freezing conditions

| | |
|---|---|
| Shelf Temperature | −50° C. |
| Ramp rate | 2.5° C./min |
| Hold time | 60 min |
| Final Freeze set point | −50° C. |
| Extra freeze time | 300 min |
| Vacuum start set point | 20 mTorr |

Primary Freeze Drying

| | |
|---|---|
| Shelf set point | −27° C. |
| Ramp Rate | 0.1° C./min |
| Time | 3600 min |
| Vacuum set point | 100 milliTorr |
| Shelf set point | −20° C. |
| Ramp Rate | 0.1° C./min |
| Time | 480 min |
| Vacuum set point | 100 milliTorr |

Secondary Drying

| | |
|---|---|
| Shelf set point | 5° C. |
| Ramp rate | 0.5° C./min |
| Time | 480 min |
| Vacuum set point | 100 milli Torr |

Final Hold

| | |
|---|---|
| Shelf set point | 5° C. |
| Time | 60 min |
| Vacuum set point | 100 milli Torr |

Various batches of liposomal topotecan suspension samples having external pH adjusted to either pH 6 or pH 4 were lyophilized using the above cycle.

### Example 5

The liposomal topotecan lyophiles were stored under various conditions, such as −15, 5 and 25° C. These samples were periodically withdrawn at 1, 2 and 3 months and were analyzed. The particle size of the original liposome suspensions before lyophilization, and the corresponding product after reconstitution of the lyophile were measured by dynamic light scattering technique using a Nicomp 380 particle size analyzer. An HPLC method is used for the determination of total and unencapsulated topotecan. The method uses an isocratic elution for the quantitation of topotecan, followed by a gradient protocol for the analysis of impurities. Mobile phase A consists of acetonitrile/water/trifluoroacetic acid in a ratio of 1/9/0.01, whereas mobile phase B is a mixture of acetonitrile/water/trifluoroacetic acid in a ratio of 4/6/0.01. The analysis is performed on a Cosmosil C18 column (or validated equivalent), and the UV detection is at 228 nm. The sample preparation used to determine total topotecan content and impurities is performed by dissolving the liposomal topotecan sample in a diluent medium consisting of methanol and trifluoroacetic acid in a ratio of 100/0.022. Unencapsulated topotecan is isolated by passing the liposomal topotecan through a centricon filter having a 30K molecular weight cut off; the filtrate is then analyzed.

According to the storage stability data presented in the Table 3 & 4, there is no significant change of the unencapsulated topotecan levels, topotecan degradants and particle size of the reconstituted lyophile after storage at various conditions for up to 3 months.

TABLE 3
Stability Summary for Liposomal Topotecan (pH 4) Lophilized

| | | 1-month | | | 2-month | | | 3-month | |
|---|---|---|---|---|---|---|---|---|---|
| Assay | Initial | −15° C. | 5° C. | 25° C. | −15° C. | 5° C. | 25° C. | 5° C. | 25° C. |
| pH | 3.9 | 3.8 | 3.8 | 3.9 | NA | NA | NA | NA | NA |
| Reconstitution | 15 | 15 | 15 | 15 | 15 | 15 | 15 | NA | NA |
| Time (Sec.) | | | | | | | | | |
| Osmolality | 385 | 393 | 394 | 393 | NA | NA | NA | NA | NA |
| (mmol/Kg) | | | | | | | | | |
| Particle Size | 112 | 111 | 112 | 113 | NA | NA | NA | NA | NA |
| (nm): Mean | | | | | | | | | |
| 25% | 82 | 77 | 76 | 76 | NA | NA | NA | NA | NA |
| 90% | 145 | 150 | 156 | 159 | NA | NA | NA | NA | NA |
| Total Topotecan | 4.08 | 4.16 | 4.16 | 4.12 | 4.12 | 4.14 | 4.10 | 4.14 | 4.15 |
| (mg/mL) | | | | | | | | | |
| Unencapsulated | 9.8 | 8.6 | 9.0 | 8.8 | 8.5 | 7.8 | 8.8 | 8.7 | 9.2 |
| Topotecan (%) | | | | | | | | | |
| Topotecan Impurity | | | | | | | | | |
| (Peak Area %): | | | | | | | | | |
| 10-hydroxy | 0.08 | 0.08 | 0.08 | 0.08 | 0.09 | 0.09 | 0.09 | 0.09 | 0.09 |
| camptothecin | | | | | | | | | |
| Dimer | <0.05 | <0.05 | <0.05 | <0.05 | <0.05 | <0.05 | <0.05 | <0.05 | <0.05 |
| Moisture (%) | 3.61 | 3.44 | 3.50 | 4.12 | NA | NA | NA | NA | NA |

TABLE 4
Stability Summary for Liposomal Topotecan (pH 6) Lophilized

| | | 1-month | | | 2-month | | | 3-month | |
|---|---|---|---|---|---|---|---|---|---|
| Assay | Initial | −15° C. | 5° C. | 25° C. | −15° C. | 5° C. | 25° C. | 5° C. | 25° C. |
| pH | 5.5 | 5.1 | 5.4 | 5.4 | NA | NA | NA | NA | NA |
| Reconstitution | 15 | 15 | 15 | 15 | 15 | 15 | 15 | NA | NA |
| Time (Sec.) | | | | | | | | | |
| Osmolality | 382 | 372 | 310 | 371 | NA | NA | NA | NA | NA |
| (mmol/Kg) | | | | | | | | | |
| Particle Size | 112 | 113 | 115 | 114 | NA | NA | NA | NA | NA |
| (nm): Mean | | | | | | | | | |
| 25% | 82 | 79 | 80 | 79 | NA | NA | NA | NA | NA |
| 90% | 145 | 154 | 158 | 156 | NA | NA | NA | NA | NA |
| Total Topotecan | 4.16 | 4.04 | 3.52 | 4.01 | 4.20 | 4.21 | 4.20 | 4.22 | 4.14 |
| (mg/mL) | | | | | | | | | |
| Unencapsulated | 7.1 | 6.5 | 8.4 | 8.4 | 5.3 | 5.7 | 6.2 | 6.3 | 7.8 |
| Topotecan (%) | | | | | | | | | |
| Topotecan Impurity | | | | | | | | | |
| (Peak Area %): | | | | | | | | | |
| 10-hydroxy | 0.10 | 0.09 | 0.10 | 0.10 | 0.10 | 0.10 | 0.09 | 0.09 | 0.09 |
| camptothecin | | | | | | | | | |
| Dimer | <0.05 | <0.05 | <0.05 | <0.05 | <0.05 | <0.05 | <0.05 | <0.05 | <0.05 |
| Moisture | 3.20 | 3.30 | 3.88 | 3.59 | NA | NA | NA | NA | NA |
| Content (%) | | | | | | | | | |

Tables 5 and 6 show the data from the needle shaped particlulate matter count for the liposomal topotecan suspension formulation at pH 6 and its corresponding lyophile respectively, during storage at various conditions. The data from Table 5 show that liposomal topotecan suspension formulation does not pass USP particulate matter test due to the formation of needle shaped crystals when stored at either 5° C. for 3 months or 25° C. for 1 month. When the same formulation is lyophilized and stored at either 5 or 25° C., there is no change in the count of the needle shaped crystals and hence the lyophilized formulations pass the USP <788> particulate matter test.

TABLE 5
Total Count of Needle shaped Particles Produced During
the Storage of Liposomal Topotecan Suspension
Formulation (pH 6) at Various Conditions.

| | Total Needle | |
|---|---|---|
| | Shaped | |
| | Particulates per | Total Needle Shaped |
| | container | Particulates per container |
| Storage Condition | (10 μm to 24 μm) | (NLT 25 μm) |
| 0 time/5° C. | 23 | 14 |
| Two weeks/5° C. | 83 | 27 |
| 1 month/5° C. | 44 | 22 |
| 2 months/5° C. | 300 | 112 |
| 3 months/5° C. | 2626 | 1065 |
| Two weeks/25° C. | 406 | 290 |
| 1 month/25° C. | TNTC* | TNTC* |
| 2 months/25° C. | TNTC* | TNTC* |

*Too numerous to count.

TABLE 6
Total Count of Needle shaped Particles Produced During the
Storage of Liposomal Topotecan Lyophile Formulation
(pH 6) at Various Conditions.

| | Total Needle Shaped | Total Needle Shaped |
|---|---|---|
| | Particulates per | Particulates per |
| | container | container |
| Storage Condition | (10 μm to 24 μm) | (NLT 25 μm) |
| 0 time/5° C. | 23 | 7 |
| 1 month/5° C. | 42 | 15 |
| 2 months/5° C. | 47 | 8 |
| 3 months/5° C. | 38 | 12 |
| 1 month/25° C. | 23 | 3 |
| 2 months/25° C. | 26 | 7 |
| 3 months/25° C. | 44 | 9 |

### Example 6

Cryo-TEM Analysis

Samples of a liposomal topotecan suspension and a lyophile from Example 2 were subjected to cryo-TEM analysis to determine whether liposome integrity is affected by the lyophilization process.

A. A sample of a liposomal topotecan suspension is diluted 1:1 with 300 mM sucrose buffer and warmed to room temperature (˜22° C.) prior to vitrification. A 4 μl aliquot of the sample is pipetted on to a holey carbon-formvar film on a 400 mesh copper grid which is been freshly glow-discharged.

The grid is gripped by forceps held closed by a rubber ring. The forceps are immediately attached, grid down, to a nitrogen gas driven plunger located in a Plexiglas freezing station. The freezing station is constantly humidified; a light fog may form but sufficient visibility to work can be maintained. A few inches below the grid liquid ethane is placed in a metal cup immersed in liquid nitrogen. The aliquot of sample on the grid is blotted for three seconds on both sides of the grid with a piece of folded filter paper mounted in a pair of wide-tipped forceps. Immediately after blotting, the grid is plunged into the supercooled ethane by activating the gas driven plunger. The vitrified sample is transferred to a small grid box immersed in liquid nitrogen. Grid boxes aree stored under liquid nitrogen in a cryogenic storage dewar.

The vitrified sample is transferred into a Gatan single tilt cryoholder using a cryotransfer station and is maintained at or near liquid nitrogen temperature during observation in a Philips CM-12 Transmission Electron Microscope. The image can be recorded on Kodak SO-163 electron image film under low dose conditions at a 1.2μ defocus. The electron micrograph can be digitized at 1270 dpi on a CreoScitex EverSmart Supreme Scanner.

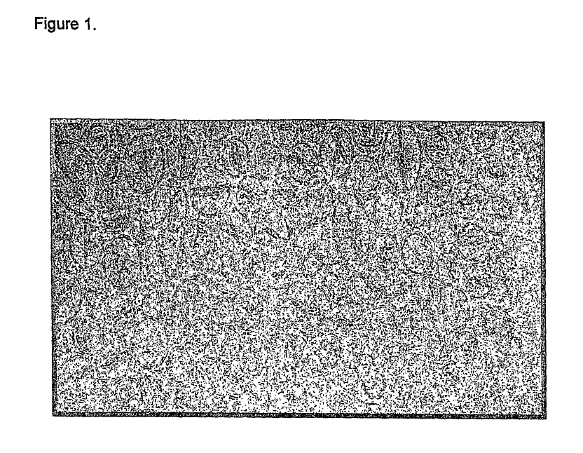

The cryo-TEM image of a sample of a liposomal topotecan suspension is provided in FIG. 1. The sample was obtained from a large scale preparation conducted in accordance with Example 1, except that the concentration of the ethanolic solution of cholesterol and sphingomyelin used was half of that described in Example 1 (e.g., on a relative scale: conducting Example 1 using 15 mL of an ethanolic solution of 0.6 g cholesterol and 1.3 g and 100 mL of hydration buffer).

B. A lyophile from Example 2 was reconstituted with 1 mL of milliQ water and sample was analyzed as follows: The sample was diluted 1:1 with 300 mM sucrose buffer and warmed to room temperature (˜22° C.) prior to vitrification. A 4 μl aliquot of the sample was pipetted on to a holey carbon-formvar film on a 400 mesh copper grid which had been freshly glow-discharged.

The grid was gripped by forceps held closed by a rubber ring. The forceps were immediately attached, grid down, to a nitrogen gas driven plunger located in a Plexiglas freezing station. The freezing station was constantly humidified; a light fog formed but sufficient visibility to work was maintained. A few inches below the grid liquid ethane was placed in a metal cup immersed in liquid nitrogen. The aliquot of sample on the grid was blotted for three seconds on both sides of the grid with a piece of folded filter paper mounted in a pair of wide-tipped forceps. Immediately after blotting, the grid was plunged into the supercooled ethane by activating the gas driven plunger. The vitrified sample was transferred to a small grid box immersed in liquid nitrogen. Grid boxes were stored under liquid nitrogen in a cryogenic storage dewar.

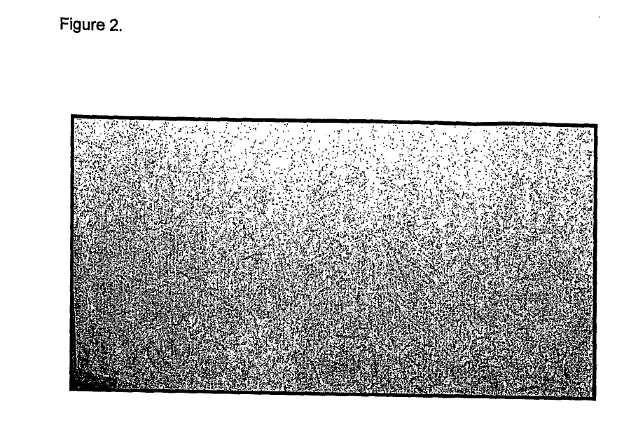

The vitrified sample was transferred into a Gatan single tilt cryoholder using a cryotransfer station and maintained at or near liquid nitrogen temperature during observation in a Philips CM-12 Transmission Electron Microscope. The image was recorded on Kodak SO-163 electron image film under low dose conditions at a 1.2μ defocus. The electron micrograph was digitized at 1270 dpi on a CreoScitex EverSmart Supreme Scanner. The cryo-TEM image of the lyophilized sample after econstitution is provided in FIG. 2.

Comparison of the cryo-TEM images of the samples taken before (FIG. 1) and after (FIG. 2) lyophilization/reconstitution show that topotecan crystals inside the liposomes are not affected by lyophilization, and that the liposome structure is maintained intact during the process of lyophilization and reconstitution.

Although the invention has been described with respect to particular formulations, preparation conditions, and uses, it will be appreciated that various modifications or changes may be made without departing from the invention. Those skilled in the art will recognize through routine experimentation that various changes and modifications can be made without departing from the scope of this invention. The various references to journals, patents and other patent applications that are cited herein are each incorporated by reference herein in their entirety, as though fully set forth.

1. A lyophilized topotecan liposomal composition comprising topotecan, liposomes and cryprotectant, wherein topotecanis substantially entrapped in the liposome interior.

2. A lyophilized topotecan liposomal composition comprising topotecan, liposomes and cryprotectant, that can be reconstituted with water to form a reconstituted composition wherein at least about 85% of the topotecan in the composition is liposome-entrapped topotecan, and wherein about 85% of the liposomes in said composition have sizes of about 0.05 to about 0.25 microns.

3. A reconstituted lyophilized topotecan liposomal composition comprising topotecan, liposomes and cryprotectant, wherein:
about 85% of the liposomes in said composition have sizes of about 0.05 to about 0.25 microns,
at least about 85% of the topotecan in the composition is liposome-entrapped topotecan; and
the topotecan/lipid ratio is between about 0.02 to about 0.4 by weight, wherein the lipid contituents comprise cholesterol and a vesicle-forming lipid that form the liposome.

4. A lyophilized topotecan liposomal composition according to claim 1, comprising no more than 2% topotecan degradation products.

5. A lyophilized topotecan liposomal composition according claim 1, which upon reconstitution after storage for an extended period of time produces no significant increase in the number and size of needle shaped particulate matter.

6. A lyophilized toptecan liposomal composition according to claim 1, wherein said cryoprotectant comprises one or more of sucrose, trehalose and lactose.

7. A process of preparing an aqueous liposomal topotecan composition suitable for injection, wherein said composition is comprised of at least about 85% liposome-entrapped topotecan, said process comprising;
reconstituting the lyophilized composition according to claim 1 to provide said aqueous composition.

8. A process of preparing an aqueous liposomal topotecan composition suitable for injection, wherein said composition is comprised of at least about 85% liposome-entrapped topotecan, said process comprising:
reconstituting the lyophilized composition according to claim 1 to form a topotecan liposomal concentrate, and
diluting the topotecan liposomal concentrate with an aqueous medium to provide said aqueous composition having a liposome concentration suitable for intravenous injection.
